(19) United States
(12) Patent Application Publication
(10) Pub. No.: US 2017/0152209 A1
(43) Pub. Date: Jun. 1, 2017

(54) ULTRA HIGH-VISCOSITY ESTOLIDE BASE OILS AND METHOD OF MAKING THE SAME

(71) Applicant: BIOSYNTHETIC TECHNOLOGIES, LLC., Irvine, CA (US)

(72) Inventors: Marlon LUTZ, Grayslake, IL (US); Travis THOMPSON, Anaheim, CA (US)

(21) Appl. No.: 15/354,826

(22) Filed: Nov. 17, 2016

Related U.S. Application Data
(60) Provisional application No. 62/261,174, filed on Nov. 30, 2015.

(51) Int. Cl. C07C 69/675; C10M 129/72

(52) U.S. Cl. CPC C07C 69/675; C10M 129/72; C10M 2207/2825; C10N 2240/04; C10N 2230/02

(57) ABSTRACT

Provided herein are methods of making estolide base oils, including those having a high kinematic viscosity. In certain embodiments, an ultra high-viscosity estolide base oil is prepared from hydroxy fatty acids derived from the hydrolysis of a first estolide base oil.

### CROSS REFERENCE TO RELATED APPLICATIONS

This application claims the benefit under 35 U.S.C. §119(e) of U.S. Provisional Patent Application No. 62/261,174, filed Nov. 30, 2015, which is incorporated herein by reference in its entirety for all purposes.

### FIELD

The present disclosure relates to estolide base oils and lubricants and methods of making the same. Exemplary estolides described herein may exhibit high-viscosity characteristics that make them suitable for use in certain applications.

### BACKGROUND

Synthetic esters such as polyol esters and adipates, poly alpha olefins (PAOs), and vegetable oils have been described for use industrially as biodegradable base stocks to formulate lubricants. Such base stocks may be used in the production of lubricating oils for automotives, industrial lubricants, and lubricating greases. Finished lubricants typically comprise the base oil and additives to help achieve desired viscometric properties, low temperature behavior, oxidative stability, corrosion protection, demulsibility and water rejection, friction coefficients, lubricities, wear protection, air release, color and other properties. However, it is generally understood that biodegradability cannot be improved by using common additives that are available in today's marketplace. Additionally, certain desired high-viscosity characteristics are difficult, if not impossible, to meet using such base stocks. Accordingly, there remains a need to develop bio-based oils and lubricants that are biodegradable and exhibit high-viscosity characteristics.

### SUMMARY

Described herein are estolide compounds, estolide-containing compositions, and methods of making the same. In certain embodiments, such compounds and/or compositions may be useful as base oils and lubricants. In certain embodiments, the estolides described herein exhibit high- and ultra high-viscometric characteristics that make them suitable for certain specialty applications.

In certain embodiments, the estolides comprise at least one compound of Formula I:

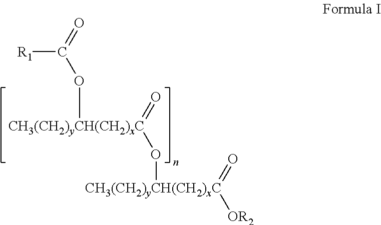

wherein

x is, independently for each occurrence, an integer selected from 0, 1, 2, 3, 4, 5, 6, 7, 8, 9, 10, 11, 12, 13, 14, 15, 16, 17, 18, 19, and 20;

y is, independently for each occurrence, an integer selected from 0, 1, 2, 3, 4, 5, 6, 7, 8, 9, 10, 11, 12, 13, 14, 15, 16, 17, 18, 19, and 20;

n is equal to or greater than 0;

R₁ is an optionally substituted alkyl that is saturated or unsaturated, and branched or unbranched; and

R₂ is selected from hydrogen and optionally substituted alkyl that is saturated or unsaturated, and branched or unbranched;
wherein each fatty acid chain residue of said at least one compound is independently optionally substituted.

In certain embodiments, the estolides comprise at least one compound of Formula II:

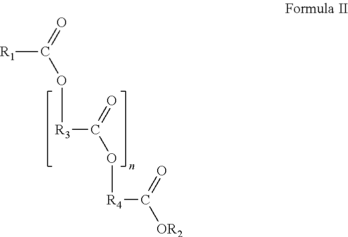

wherein

n is an integer equal to or greater than 0;

R₁ is an optionally substituted alkyl that is saturated or unsaturated, and branched or unbranched; and

R₂ is selected from hydrogen and optionally substituted alkyl that is saturated or unsaturated, and branched or unbranched; and

R₃ and R₄, independently for each occurrence, are selected from optionally substituted alkyl that is saturated or unsaturated, and branched or unbranched.

### DETAILED DESCRIPTION

The use of lubricants and lubricant-containing compositions may result in the dispersion of such fluids, compounds, and/or compositions in the environment. Petroleum base oils used in common lubricant compositions, as well as additives, are typically non-biodegradable and can be toxic. The present disclosure provides for the preparation and use of compositions comprising partially or fully biodegradable base oils, including base oils comprising one or more estolides.

In certain embodiments, the compositions comprising one or more estolides are partially or fully biodegradable and thereby pose diminished risk to the environment. In certain embodiments, the compositions meet guidelines set for by the Organization for Economic Cooperation and Development (OECD) for degradation and accumulation testing. The OECD has indicated that several tests may be used to determine the “ready biodegradability” of organic chemicals. Aerobic ready biodegradability by OECD 301D measures the mineralization of the test sample to CO₂ in closed aerobic microcosms that simulate an aerobic aquatic environment, with microorganisms seeded from a waste-water treatment plant. OECD 301D is considered representative of most aerobic environments that are likely to receive waste materials. Aerobic “ultimate biodegradability” can be determined by OECD 302D. Under OECD 302D, microorganisms are pre-acclimated to biodegradation of the test material during a pre-incubation period, then incubated in sealed vessels with relatively high concentrations of microorganisms and enriched mineral salts medium. OECD 302D ultimately determines whether the test materials are completely biodegradable, albeit under less stringent conditions than “ready biodegradability” assays.

As used in the present specification, the following words, phrases and symbols are generally intended to have the meanings as set forth below, except to the extent that the context in which they are used indicates otherwise. The following abbreviations and terms have the indicated meanings throughout:

A dash (“—”) that is not between two letters or symbols is used to indicate a point of attachment for a substituent. For example, —C(O)NH₂ is attached through the carbon atom.

“Alkoxy” by itself or as part of another substituent refers to a radical —OR³¹ where R³¹ is alkyl, cycloalkyl, cycloalkylalkyl, aryl, or arylalkyl, which can be substituted, as defined herein. In some embodiments, alkoxy groups have from 1 to 8 carbon atoms. In some embodiments, alkoxy groups have 1, 2, 3, 4, 5, 6, 7, or 8 carbon atoms. Examples of alkoxy groups include, but are not limited to, methoxy, ethoxy, propoxy, butoxy, cyclohexyloxy, and the like.

“Alkyl” by itself or as part of another substituent refers to a saturated or unsaturated, branched, or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1 -yl, but-1-yn-3-yl, but-3-yn-1-yl, etc.; and the like.

Unless otherwise indicated, the term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group comprises from 1 to 40 carbon atoms, in certain embodiments, from 1 to 22 or 1 to 18 carbon atoms, in certain embodiments, from 1 to 16 or 1 to 8 carbon atoms, and in certain embodiments from 1 to 6 or 1 to 3 carbon atoms. In certain embodiments, an alkyl group comprises from 8 to 22 carbon atoms, in certain embodiments, from 8 to 18 or 8 to 16. In some embodiments, the alkyl group comprises from 3 to 20 or 7 to 17 carbons. In some embodiments, the alkyl group comprises 1, 2, 3, 4, 5, 6, 7, 8, 9, 10, 11, 12, 13, 14, 15, 16, 17, 18, 19, 20, 21, or 22 carbon atoms.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered non-aromatic heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can comprise from 5 to 20 carbon atoms, and in certain embodiments, from 5 to 12 carbon atoms. In certain embodiments, an aryl group can comprise 5, 6, 7, 8, 9, 10, 11, 12, 13, 14, 15, 16, 17, 18, 19, or 20 carbon atoms. Aryl, however, does not encompass or overlap in any way with heteroaryl, separately defined herein. Hence, a multiple ring system in which one or more carbocyclic aromatic rings is fused to a heterocycloalkyl aromatic ring, is heteroaryl, not aryl, as defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

Estolide “base oil” and “base stock”, unless otherwise indicated, refer to any composition comprising one or more estolide compounds. It should be understood that an estolide “base oil” or “base stock” is not limited to compositions for a particular use, and may generally refer to compositions comprising one or more estolides, including mixtures of estolides. Estolide base oils and base stocks can also include compounds other than estolides.

“Compounds” refers to compounds encompassed by structural Formula I and II herein and includes any specific compounds within the formula whose structure is disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

For the purposes of the present disclosure, “chiral compounds” are compounds having at least one center of chirality (i.e. at least one asymmetric atom, in particular at least one asymmetric C atom), having an axis of chirality, a plane of chirality or a screw structure. “Achiral compounds” are compounds which are not chiral.

Compounds of Formula I and II include, but are not limited to, optical isomers of compounds of Formula I and II, racemates thereof, and other mixtures thereof. In such embodiments, the single enantiomers or diastereomers, i.e., optically active forms, can be obtained by asymmetric synthesis or by resolution of the racemates. Resolution of the racemates may be accomplished by, for example, chromatography, using, for example a chiral high-pressure liquid chromatography (HPLC) column. However, unless otherwise stated, it should be assumed that Formula I and II cover all asymmetric variants of the compounds described herein, including isomers, racemates, enantiomers, diastereomers, and other mixtures thereof. In addition, compounds of Formula I and II include Z- and E-forms (e.g., cis- and trans-forms) of compounds with double bonds. The compounds of Formula I and II may also exist in several tautomeric forms including the enol form, the keto form, and mixtures thereof. Accordingly, the chemical structures depicted herein encompass all possible tautomeric forms of the illustrated compounds.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₃₋₁₂ cycloalkyl or C₅₋₁₂ cycloalkyl. In certain embodiments, a cycloalkyl group is a C₅, C₆, C₇, C₈, C₉, C₁₀, C₁₁, C₁₂, C₁₃, C₁₄, or C₁₅ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂.

“Halogen” refers to a fluoro, chloro, bromo, or iodo group.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which can be aromatic or non-aromatic in which at least one ring atom is a heteroatom. Heteroaryl encompasses 5- to 12-membered aromatic, such as 5- to 7-membered, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 12-membered heteroaryl or from 5- to 10-membered heteroaryl. In certain embodiments, a heteroaryl group is a 5-, 6-, 7-, 8-, 9-, 10-, 11-, 12-, 13-, 14-, 15-, 16-, 17-, 18-, 19-, or 20-membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Heterocycloalkyl” by itself or as part of another substituent refers to a partially saturated or unsaturated cyclic alkyl radical in which one or more carbon atoms (and any associated hydrogen atoms) are independently replaced with the same or different heteroatom. Examples of heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like.

“Heterocycloalkylalkyl” by itself or as part of another substituent refers to an acyclic alkyl radical in which one of the hydrogen atoms bonded to a carbon atom, typically a terminal or sp³carbon atom, is replaced with a heterocycloalkyl group. Where specific alkyl moieties are intended, the nomenclature heterocycloalkylalkanyl, heterocycloalkylalkenyl, or heterocycloalkylalkynyl is used. In certain embodiments, a heterocycloalkylalkyl group is a 6- to 30-membered heterocycloalkylalkyl, e.g., the alkanyl, alkenyl, or alkynyl moiety of the heterocycloalkylalkyl is 1- to 10-membered and the heterocycloalkyl moiety is a 5- to 20-membered heterocycloalkyl, and in certain embodiments, 6- to 20-membered heterocycloalkylalkyl, e.g., the alkanyl, alkenyl, or alkynyl moiety of the heterocycloalkylalkyl is 1- to 8-membered and the heterocycloalkyl moiety is a 5- to 12-membered heterocycloalkyl.

“Mixture” refers to a collection of molecules or chemical substances. Each component in a mixture can be independently varied. A mixture may contain, or consist essentially of, two or more substances intermingled with or without a constant percentage composition, wherein each component may or may not retain its essential original properties, and where molecular phase mixing may or may not occur. In mixtures, the components making up the mixture may or may not remain distinguishable from each other by virtue of their chemical structure.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Substituted” refers to a group in which one or more hydrogen atoms are independently replaced with the same or different substituent(s). Examples of substituents include, but are not limited to, —R⁶⁴, —R⁶⁰, —O⁻, ═OH, ═O, —OR⁶⁰, —SR⁶⁰, —S⁻, ═S, —NR⁶⁰R⁶¹, ═NR⁶⁰, —CN, —CF₃, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R₆₁, —NR⁶²C(S)NR⁶⁰R⁶¹, —NR⁶²C(NR⁶³)NR⁶⁰R⁶¹, —C(NR⁶²)NR⁶⁰R⁶¹, —S(O)₂, NR⁶⁰R⁶¹, —NR⁶³S(O)₂R⁶⁰, —NR⁶³C(O)R⁶⁰, and —S(O)R⁶⁰;

wherein each —R⁶⁴ is independently a halogen; each R⁶⁰ and R⁶¹ are independently alkyl, substituted alkyl, alkoxy, substituted alkoxy, cycloalkyl, substituted cycloalkyl, heterocycloalkyl, substituted heterocycloalkyl, aryl, substituted aryl, heteroaryl, substituted heteroaryl, arylalkyl, substituted arylalkyl, heteroarylalkyl, or substituted heteroarylalkyl, or R⁶⁰ and R⁶¹ together with the nitrogen atom to which they are bonded form a heterocycloalkyl, substituted heterocycloalkyl, heteroaryl, or substituted heteroaryl ring, and R⁶² and R⁶³ are independently alkyl, substituted alkyl, aryl, substituted aryl, arylalkyl, substituted arylalkyl, cycloalkyl, substituted cycloalkyl, heterocycloalkyl, substituted heterocycloalkyl, heteroaryl, substituted heteroaryl, heteroarylalkyl, or substituted heteroarylalkyl, or R⁶² and R⁶³ together with the atom to which they are bonded form one or more heterocycloalkyl, substituted heterocycloalkyl, heteroaryl, or substituted heteroaryl rings;

wherein the “substituted” substituents, as defined above for R⁶⁰, R⁶¹, R⁶², and R⁶³, are substituted with one or more, such as one, two, or three, groups independently selected from alkyl, —alkyl-OH, —O-haloalkyl, -alkyl-NH₂, alkoxy, cycloalkyl, cycloalkylalkyl, heterocycloalkyl, heterocycloalkylalkyl, aryl, heteroaryl, arylalkyl, heteroarylalkyl, —O⁻, —OH, ═O, —O-alkyl, —O-aryl, —O-heteroarylalkyl, —O-cycloalkyl, —O-heterocycloalkyl, —SH, —S³¹ , ═S, —S-alkyl, —S-aryl, —S-heteroarylalkyl, —S-cycloalkyl, —S-heterocycloalkyl, —NH₂, ═NH, —CN, —CF₃, —OCN, —SCN, —NO, —NO₂, ═N₂, —N₃, —S(O)₂O⁻, —S(O)₂, —S(O)₂OH, —OS(O₂)O⁻, —SO₂(alkyl), —SO₂(phenyl), —SO₂(haloalkyl), —SO₂NH₂, —SO₂NH(alkyl), —SO₂NH(phenyl), —P(O)O⁻)₂, —P(O)(O-alkyl)(O⁻), —OP(O)(O-alkyl)(O-alkyl), —CO₂H, —C(O)O(alkyl), —CON(alkyl)(alkyl), —CONH(alkyl), —CONH₂, —C(O)(alkyl), —C(O)(phenyl), —C(O)(haloalkyl), —OC(O)(alkyl), —N(alkyl)(alkyl), —NH(alkyl), —N(alkyl)(alkylphenyl), —NH(alkylphenyl), —NHC(O)(alkyl), —NHC(O)(phenyl), —N(alkyl)C(O)(alkyl), and —N(alkyl)C(O)(phenyl).

As used in this specification and the appended claims, the articles “a,” “an,” and “the” include plural referents unless expressly and unequivocally limited to one referent.

All numerical ranges herein include all numerical values and ranges of all numerical values within the recited range of numerical values.

The present disclosure relates to estolide compounds, compositions and methods of making the same. In certain embodiments, the present disclosure also relates to estolide compounds, and compositions comprising estolide compounds, for high- and ultra high-viscosity base oil stocks and lubricants, the synthesis of such compounds, and the formulation of such compositions. In certain embodiments, the present disclosure relates to biosynthetic estolides having desired viscometric properties, while retaining or even improving other properties such as oxidative stability and pour point. In certain embodiments, new methods of preparing estolide compounds exhibiting such properties are provided. The present disclosure also relates to compositions comprising certain estolide compounds exhibiting such properties.

In certain embodiments the composition comprises at least one estolide compound of Formula I:

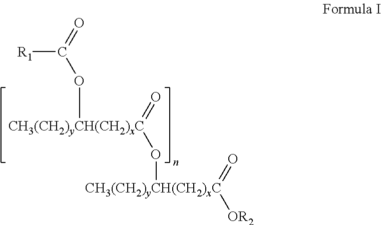

wherein

x is, independently for each occurrence, an integer selected from 0, 1, 2, 3, 4, 5, 6, 7, 8, 9, 10, 11, 12, 13, 14, 15, 16, 17, 18, 19, and 20;

y is, independently for each occurrence, an integer selected from 0, 1, 2, 3, 4, 5, 6, 7, 8, 9, 10, 11, 12, 13, 14, 15, 16, 17, 18, 19, and 20;

n is equal to or greater than 0;

R₁ is an optionally substituted alkyl that is saturated or unsaturated, and branched or unbranched; and

R₂ is selected from hydrogen and optionally substituted alkyl that is saturated or unsaturated, and branched or unbranched;

wherein each fatty acid chain residue of said at least one compound is independently optionally substituted.

In certain embodiments the composition comprises at least one estolide compound of Formula II:

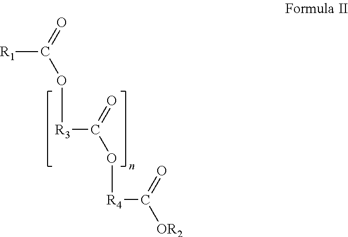

wherein

n is an integer equal to or greater than 0;

R₁ is an optionally substituted alkyl that is saturated or unsaturated, and branched or unbranched; and

R₂ is selected from hydrogen and optionally substituted alkyl that is saturated or unsaturated, and branched or unbranched; and

R₃ and R₄, independently for each occurrence, are selected from optionally substituted alkyl that is saturated or unsaturated, and branched or unbranched.

In certain embodiments, the composition comprises at least one estolide of Formula I or II where R₁ is hydrogen.

The terms “chain” or “fatty acid chain” or “fatty acid chain residue,” as used with respect to the estolide compounds of Formula I and II, refer to one or more of the fatty acid residues incorporated in estolide compounds, e.g., R₃ or R₄ of Formula II, or the structures represented by CH₃(CH₂)_{y}CH(CH₂)ₓC(O)O— in Formula I.

The R₁ in Formula I and II at the top of each Formula shown is an example of what may be referred to as a “cap” or “capping material,” as it “caps” the top of the estolide. Similarly, the capping group may be an organic acid residue of general formula —OC(O)-alkyl, i.e., a carboxylic acid with a substituted or unsubstituted, saturated or unsaturated, and/or branched or unbranched alkyl as defined herein, or a formic acid residue. In certain embodiments, the “cap” or “capping group” is a fatty acid. In certain embodiments, the capping group, regardless of size, is substituted or unsubstituted, saturated or unsaturated, and/or branched or unbranched. The cap or capping material may also be referred to as the primary or alpha (α) chain.

Depending on the manner in which the estolide is synthesized, the cap or capping group alkyl may be the only alkyl from an organic acid residue in the resulting estolide that is unsaturated. In certain embodiments, it may be desirable to use a saturated organic or fatty-acid cap to increase the overall saturation of the estolide and/or to increase the resulting estolide's stability. For example, in certain embodiments, it may be desirable to provide a method of providing a saturated capped estolide by hydrogenating an unsaturated cap using any suitable methods available to those of ordinary skill in the art. Hydrogenation may be used with various sources of the fatty-acid feedstock, which may include mono- and/or polyunsaturated fatty acids. Without being bound to any particular theory, in certain embodiments, hydrogenating the estolide may help to improve the overall stability of the molecule. However, a fully-hydrogenated estolide, such as an estolide with a larger fatty acid cap, may exhibit increased pour point temperatures. In certain embodiments, it may be desirable to offset any loss in desirable pour-point characteristics by using shorter, saturated capping materials.

The R₄C(O)O— of Formula II or structure CH₃(CH₂)_{y}CH(CH₂)ₓC(O)O— of Formula I serve as the “base” or “base chain residue” of the estolide. Depending on the manner in which the estolide is synthesized, the base organic acid or fatty acid residue may be the only residue that remains in its free-acid form after the initial synthesis of the estolide. However, in certain embodiments, in an effort to alter or improve the properties of the estolide, the free acid may be reacted with any number of substituents. For example, it may be desirable to react the free acid estolide with alcohols, glycols, amines, or other suitable reactants to provide the corresponding ester, amide, or other reaction products. The base or base chain residue may also be referred to as tertiary or gamma (γ) chains.

The R₃C(O)O— of Formula II or structure CH₃(CH₂)_{y}CH(CH₂)ₓC(O)O— of Formula I are linking residues that link the capping material and the base fatty-acid residue together. There may be any number of linking residues in the estolide, including when n=0 and the estolide is in its dimer form. Depending on the manner in which the estolide is prepared, a linking residue may be a fatty acid and may initially be in an unsaturated form during synthesis. In some embodiments, the estolide will be formed when a catalyst is used to produce a carbocation at the fatty acid's site of unsaturation, which is followed by nucleophilic attack on the carbocation by the carboxylic group of another fatty acid. In some embodiments, it may be desirable to have a linking fatty acid that is monounsaturated so that when the fatty acids link together, all of the sites of unsaturation are eliminated. The linking residue(s) may also be referred to as secondary or beta ((β) chains.

In certain embodiments, the cap is an acetyl group, the linking residue(s) is one or more fatty acid residues, and the base chain residue is a fatty acid residue. In certain embodiments, the linking residues present in an estolide differ from one another. In certain embodiments, one or more of the linking residues differs from the base chain residue.

As noted above, in certain embodiments, suitable unsaturated fatty acids for preparing the estolides may include any mono- or polyunsaturated fatty acid. For example, monounsaturated fatty acids, along with a suitable catalyst, will form a single carbocation that allows for the addition of a second fatty acid, whereby a single link between two fatty acids is formed. Suitable monounsaturated fatty acids may include, but are not limited to, palmitoleic acid (16:1), vaccenic acid (18:1), oleic acid (18:1), eicosenoic acid (20:1), erucic acid (22:1), and nervonic acid (24:1). In addition, in certain embodiments, polyunsaturated fatty acids may be used to create estolides. Suitable polyunsaturated fatty acids may include, but are not limited to, hexadecatrienoic acid (16:3), alpha-linolenic acid (18:3), stearidonic acid (18:4), eicosatrienoic acid (20:3), eicosatetraenoic acid (20:4), eicosapentaenoic acid (20:5), heneicosapentaenoic acid (21:5), docosapentaenoic acid (22:5), docosahexaenoic acid (22:6), tetracosapentaenoic acid (24:5), tetracosahexaenoic acid (24:6), linoleic acid (18:2), gamma-linoleic acid (18:3), eicosadienoic acid (20:2), dihomo-gamma-linolenic acid (20:3), arachidonic acid (20:4), docosadienoic acid (20:2), adrenic acid (22:4), docosapentaenoic acid (22:5), tetracosatetraenoic acid (22:4), tetracosapentaenoic acid (24:5), pinolenic acid (18:3), podocarpic acid (20:3), rumenic acid (18:2), alpha-calendic acid (18:3), beta-calendic acid (18:3), jacaric acid (18:3), alpha-eleostearic acid (18:3), beta-eleostearic (18:3), catalpic acid (18:3), punicic acid (18:3), rumelenic acid (18:3), alpha-parinaric acid (18:4), beta-parinaric acid (18:4), and bosseopentaenoic acid (20:5). In certain embodiments, hydroxy fatty acids may be polymerized or homopolymerized by reacting the carboxylic acid functionality of one fatty acid with the hydroxy functionality of a second fatty acid. Exemplary hydroxyl fatty acids include, but are not limited to, ricinoleic acid, 6-hydroxystearic acid, 9,10-dihydroxystearic acid, 12-hydroxystearic acid, and 14-hydroxystearic acid.

The process for preparing the estolide compounds described herein may include the use of any natural or synthetic fatty acid source. However, it may be desirable to source the fatty acids from a renewable biological feedstock. Suitable starting materials of biological origin may include plant fats, plant oils, plant waxes, animal fats, animal oils, animal waxes, fish fats, fish oils, fish waxes, algal oils and mixtures thereof. Other potential fatty acid sources may include waste and recycled food-grade fats and oils, fats, oils, and waxes obtained by genetic engineering, fossil fuel-based materials and other sources of the materials desired.

In certain embodiments, the estolide compounds described herein may be prepared from non-naturally occurring fatty acids derived from naturally occurring feedstocks. In certain embodiments, the estolides are prepared from synthetic fatty acid reactants derived from naturally occurring feedstocks such as vegetable oils. For example, the synthetic fatty acid reactants may be prepared by cleaving fragments from larger fatty acid residues occurring in natural oils such as triglycerides using, for example, a cross-metathesis catalyst and alpha-olefin(s). The resulting truncated fatty acid residue(s) may be liberated from the glycerine backbone using any suitable hydrolytic and/or transesterification processes known to those of skill in the art. An exemplary fatty acid reactant includes 9-decenoic acid, which may be prepared via the cross metathesis of an oleic acid residue with an olefin such as ethene.

In some embodiments, the estolide comprises fatty-acid chains of varying lengths. In some embodiments, x is, independently for each occurrence, an integer selected from 0 to 20, 0 to 18, 0 to 16, 0 to 14, 1 to 12, 1 to 10, 2 to 8, 6 to 8, 7 to 10, or 4 to 6. In some embodiments, x is, independently for each occurrence, an integer selected from 7 and 8. In some embodiments, x is, independently for each occurrence, an integer selected from 0, 1, 2, 3, 4, 5, 6, 7, 8, 9, 10, 11, 12, 13, 14, 15, 16, 17, 18, 19, and 20. In certain embodiments, for at least one fatty acid chain residue, x is an integer selected from 7 and 8.

In some embodiments, y is, independently for each occurrence, an integer selected from 0 to 20, 0 to 18, 0 to 16, 0 to 14, 1 to 12, 1 to 10, 2 to 8, 5 to 8, 6 to 8, or 4 to 6. In some embodiments, y is, independently for each occurrence, an integer selected from 7 and 8. In some embodiments, y is, independently for each occurrence, an integer selected from 0, 1, 2, 3, 4, 5, 6, 7, 8, 9, 10, 11, 12, 13, 14, 15, 16, 17, 18, 19, and 20. In some embodiments, for at least one fatty acid chain residue, y is an integer selected from 0 to 6, or 1 and 2. In certain embodiments, y is, independently for each occurrence, an integer selected from 1 to 6, or 1 and 2. In certain embodiments, y is 0.

In some embodiments, x+y is, independently for each chain, an integer selected from 0 to 40, 0 to 20, 10 to 20, or 12 to 18. In some embodiments, x+y is, independently for each chain, an integer selected from 13 to 15. In some embodiments, x+y is 15 for each chain. In some embodiments, x+y is, independently for each chain, an integer selected from 6, 7, 8, 9, 10, 11, 12, 13, 14, 15, 16, 17, 18, 19, 20, 21, 22, 23, and 24. In certain embodiments, for at least one fatty acid chain residue, x+y is an integer selected from 9 to 13. In certain embodiments, for at least one fatty acid chain residue, x+y is 9. In certain embodiments, x+y is, independently for each chain, an integer selected from 9 to 13. In certain embodiments, x+y is 9 for each fatty acid chain residue. In certain embodiments, x is 7 and y is 0, wherein x+y is 7.

In some embodiments, the estolide compound of Formula I or II may comprise any number of fatty acid residues to form an “n-mer” estolide. For example, the estolide may be in its dimer (n=0), trimer (n=1), tetramer (n=2), pentamer (n=3), hexamer (n=4), heptamer (n=5), octamer (n=6), nonamer (n=7), or decamer (n=8) form. In some embodiments, n is an integer selected from 0 to 20, 0 to 18, 0 to 16, 0 to 14, 0 to 12, 0 to 10, 0 to 8, or 0 to 6. In some embodiments, n is an integer selected from 0 to 4. In some embodiments, n is 1, wherein said at least one compound of Formula I or II comprises the trimer. In some embodiments, n is greater than 1. In some embodiments, n is an integer selected from 0, 1, 2, 3, 4, 5, 6, 7, 8, 9, 10, 11, 12, 13, 14, 15, 16, 17, 18, 19, and 20.

In certain embodiments, the estolide compounds and compositions described herein exhibit high- and ultra-high viscosities. In certain embodiments, such high- and ultra-high viscosity properties may be attributable to the size of the estolide oligomer, i.e., the estolide number (EN) of the estolide and the value of “n” with regard to Formula I and II. Thus, in certain embodiments, n has a value of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40. In certain embodiments, n is an integer selected from 0 to 50, 10 to 30, 10 to 50, 15 to 30, 20 to 30, or 15 to 25.

In some embodiments, R₁ of Formula I or II is an optionally substituted alkyl that is saturated or unsaturated, and branched or unbranched. In some embodiments, the alkyl group is a C₁ to C₄₀ alkyl, C₁ to C₂₂ alkyl or C₁ to C₁₈ alkyl. In some embodiments, the alkyl group is selected from C₇ to C₁₇ alkyl. In some embodiments, R₁ is selected from C₇ alkyl, C₉ alkyl, C₁₁ alkyl, C₁₃ alkyl, C₁₅ alkyl, and C₁₇ alkyl. In some embodiments, R₁ is selected from C₁₃ to C₁₇ alkyl, such as from C₁₃ alkyl, C₁₅ alkyl, and C₁₇ alkyl. In some embodiments, R₁ is a Cᵢ, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl.

In some embodiments, R₂ of Formula I or II is an optionally substituted alkyl that is saturated or unsaturated, and branched or unbranched. In some embodiments, the alkyl group is a C₁ to C₄₀ alkyl, C₁ to C₂₂ alkyl or C₁ to C₁₈ alkyl. In some embodiments, the alkyl group is selected from C₇ to C₁₇ alkyl. In some embodiments, R₂ is selected from C₇ alkyl, C₉ alkyl, C₁₁ alkyl, C₁₃ alkyl, C₁₅ alkyl, and C₁₇ alkyl. In some embodiments, R₂ is selected from C₁₃ to C₁₇ alkyl, such as from C₁₃ alkyl, C₁₅ alkyl, and C₁₇ alkyl. In some embodiments, R₂ is a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl.

In some embodiments, R₃ is an optionally substituted alkyl that is saturated or unsaturated, and branched or unbranched. In some embodiments, the alkyl group is a C₁ to C₄₀ alkyl, C₁ to C₂₂ alkyl or C₁ to C₁₈ alkyl. In some embodiments, the alkyl group is selected from C₇ to C₁₇ alkyl. In some embodiments, R₃ is selected from C₇ alkyl, C₉ alkyl, C₁₁ alkyl, C₁₃ alkyl, C₁₅ alkyl, and C₁₇ alkyl. In some embodiments, R₃ is selected from C₁₃ to C₁₇ alkyl, such as from C₁₃ alkyl, C₁₅ alkyl, and C₁₇ alkyl. In some embodiments, R₃ is a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl.

In some embodiments, R₄ is an optionally substituted alkyl that is saturated or unsaturated, and branched or unbranched. In some embodiments, the alkyl group is a C₁ to C₄₀ alkyl, C₁ to C₂₂ alkyl or C₁ to C₁₈ alkyl. In some embodiments, the alkyl group is selected from C₇ to C₁₇ alkyl. In some embodiments, R₄ is selected from C₇ alkyl, C₉ alkyl, C₁₁ alkyl, C₁₃ alkyl, C₁₅ alkyl, and C₁₇ alkyl. In some embodiments, R₄ is selected from C₁₃ to C₁₇ alkyl, such as from C₁₃ alkyl, C₁₅ alkyl, and C₁₇ alkyl. In some embodiments, R₄ is a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl.

As noted above, in certain embodiments, it may be possible to manipulate one or more of the estolides' properties by altering the length of R₁ and/or its degree of saturation. However, in certain embodiments, the level of substitution on R₁ may also be altered to change or even improve the estolides' properties. Without being bound to any particular theory, in certain embodiments, it is believed that the presence of polar substituents on R₁, such as one or more hydroxy groups, may increase the viscosity of the estolide, while increasing pour point. Accordingly, in some embodiments, R₁ will be unsubstituted or optionally substituted with a group that is not hydroxyl. In certain embodiments, the overall size of the oligomer (i.e., high EN) may minimize the effects of any hydroxyl groups present on the R₁ capping residue. Accordingly, in certain embodiments, R₁ is substituted with at least one hydroxyl group.

In some embodiments, the estolide is in its free-acid form, wherein R₂ of Formula I or II is hydrogen. In some embodiments, R₂ is selected from optionally substituted alkyl that is saturated or unsaturated, and branched or unbranched. In certain embodiments, the R₂ residue may comprise any desired alkyl group, such as those derived from esterification of the estolide with the alcohols identified in the examples herein. In some embodiments, the alkyl group is selected from C₁ to C₄₀, C₁ to C₂₂, C₃ to C₂₀, C₁ to C₁₈, or C₆ to C₁₂ alkyl. In some embodiments, R₂ may be selected from C₃ alkyl, C₄ alkyl, C₈ alkyl, C₁₂ alkyl, C₁₆ alkyl, C₁₈ alkyl, and C₂₀ alkyl. For example, in certain embodiments, R₂ may be branched, such as isopropyl, isobutyl, or 2-ethylhexyl. In some embodiments, R₂ may be a larger alkyl group, branched or unbranched, comprising C₁₂ alkyl, C₁₆ alkyl, C₁₈ alkyl, or C₂₀ alkyl. Such groups at the R₂ position may be derived from esterification of the free-acid estolide using the Jarcol™ line of alcohols marketed by Jarchem Industries, Inc. of Newark, N.J., including Jarcol™ I-18CG, I-20, I-12, I-16, I-18T, and 85BJ. In some cases, R₂ may be sourced from certain alcohols to provide branched alkyls such as isostearyl and isopalmityl. It should be understood that such isopalmityl and isostearyl akyl groups may cover any branched variation of C₁₆ and C₁₈, respectively. For example, the estolides described herein may comprise highly-branched isopalmityl or isostearyl groups at the R₂ position, derived from the Fineoxocol® line of isopalmityl and isostearyl alcohols marketed by Nissan Chemical America Corporation of Houston, Tex., including Fineoxocol® 180, 180N, and 1600. Without being bound to any particular theory, in embodiments, large, highly-branched alkyl groups (e.g., isopalmityl and isostearyl) at the R₂ position of the estolides canprovide at least one way to increase the lubricant's viscosity, while substantially retaining or even reducing its pour point.

In some embodiments, the compounds described herein may comprise a mixture of two or more estolide compounds of Formula I and II. It is possible to characterize the chemical makeup of an estolide, a mixture of estolides, or a composition comprising estolides, by using the compound's, mixture's, or composition's measured estolide number (EN) of compound or composition. The EN represents the average number of fatty acids added to the base fatty acid. The EN also represents the average number of estolide linkages per molecule:

EN=n+1

wherein n is the number of secondary ((β) fatty acids. Accordingly, a single estolide compound will have an EN that is a whole number, for example for dimers, trimers, and tetramers:

dimer EN=1

trimer EN=2

tetramer EN=3

However, a composition comprising two or more estolide compounds may have an EN that is a whole number or a fraction of a whole number. For example, a composition having a 1:1 molar ratio of dimer and trimer would have an EN of 1.5, while a composition having a 1:1 molar ratio of tetramer and trimer would have an EN of 2.5.

In some embodiments, the compositions may comprise a mixture of two or more estolides having an EN that is an integer or fraction of an integer that is greater than 4.5, or even 5.0. In some embodiments, the EN may be an integer or fraction of an integer selected from about 1.0 to about 5.0. In some embodiments, the EN is an integer or fraction of an integer selected from 1.2 to about 4.5. In some embodiments, the EN is selected from a value greater than 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6 and 5.8. In some embodiments, the EN is selected from a value less than 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, and 5.0, 5.2, 5.4, 5.6, 5.8, and 6.0. In some embodiments, the EN is selected from 1, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, and 6.0. In certain embodiments, the EN is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In certain embodiments, the EN is about 10 to about 30. In certain embodiments, the EN is about 15 to about 30. In certain embodiments, the EN is about 20 to about 40. In certain embodiments, the EN is about 20 to about 30. In certain embodiments, the EN is about 15 to about 25.

As noted above, it should be understood that the chains of the estolide compounds may be independently optionally substituted, wherein one or more hydrogens are removed and replaced with one or more of the substituents identified herein. Similarly, two or more of the hydrogen residues may be removed to provide one or more sites of unsaturation, such as a cis or trans double bond. Further, the chains may optionally comprise branched hydrocarbon residues. For example, in some embodiments the estolides described herein may comprise at least one compound of Formula II:

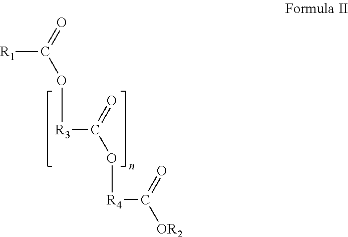

wherein
n is an integer equal to or greater than 0;
R₁ is an optionally substituted alkyl that is saturated or unsaturated, and branched or unbranched
R₂ is selected from hydrogen and optionally substituted alkyl that is saturated or unsaturated, and branched or unbranched; and
R₃ and R₄, independently for each occurrence, are selected from optionally substituted alkyl that is saturated or unsaturated, and branched or unbranched.

In some embodiments, n is an integer selected from 1 to 20. In some embodiments, n is an integer selected from 1 to 12. In some embodiments, n is an integer selected from 1, 2, 3, 4, 5, 6, 7, 8, 9, 10, 11, 12, 13, 14, 15, 16, 17, 18, 19 and 20. In certain embodiments, n is an integer selected from 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40. In certain embodiments, n is an integer selected from 10 to 30, 15 to 30, 20 to 30, or 15 to 25. In some embodiments, one or more R₃ differs from one or more other R₃ in a compound of Formula II. In some embodiments, one or more R₃ differs from R₄ in a compound of Formula II. In some embodiments, if the compounds of Formula II are prepared from one or more polyunsaturated fatty acids, it is possible that one or more of R₃ and R₄ will have one or more sites of unsaturation. In some embodiments, if the compounds of Formula II are prepared from one or more branched fatty acids, it is possible that one or more of R₃ and R₄ will be branched.

In some embodiments, R₃ and R₄ can be CH₃(CH₂)_{y}CH(CH₂)ₓ—, where x is, independently for each occurrence, an integer selected from 0, 1, 2, 3, 4, 5, 6, 7, 8, 9, 10, 11, 12, 13, 14, 15, 16, 17, 18, 19, and 20, and y is, independently for each occurrence, an integer selected from 0, 1, 2, 3, 4, 5, 6, 7, 8, 9, 10, 11, 12, 13, 14, 15, 16, 17, 18, 19, and 20. Where both R₃ and R₄ are CH₃(CH₂)_{y}CH(CH₂)ₓ—, the compounds may be compounds according to Formula I.

Without being bound to any particular theory, in certain embodiments, altering the EN produces estolides having desired viscometric properties while substantially retaining or even reducing pour point. For example, in some embodiments the estolides exhibit a decreased pour point upon increasing the EN value. Accordingly, in certain embodiments, a method is provided for retaining or decreasing the pour point of an estolide base oil by increasing the EN of the base oil, or a method is provided for retaining or decreasing the pour point of a composition comprising an estolide base oil by increasing the EN of the base oil. In some embodiments, the method comprises: selecting an estolide base oil having an initial EN and an initial pour point; and removing at least a portion of the base oil, said portion exhibiting an EN that is less than the initial EN of the base oil, wherein the resulting estolide base oil exhibits an EN that is greater than the initial EN of the base oil, and a pour point that is equal to or lower than the initial pour point of the base oil. In some embodiments, the selected estolide base oil is prepared by oligomerizing at least one first unsaturated fatty acid with at least one second unsaturated fatty acid and/or saturated fatty acid. In some embodiments, the removing at least a portion of the base oil is accomplished by distillation, chromatography, membrane separation, phase separation, affinity separation, solvent extraction, or combinations thereof. In some embodiments, the distillation takes place at a temperature and/or pressure that is suitable to separate the estolide base oil into different “cuts” that individually exhibit different EN values. In some embodiments, this may be accomplished by subjecting the base oil temperature of at least about 250° C. and an absolute pressure of no greater than about 25 microns. In some embodiments, the distillation takes place at a temperature range of about 250° C. to about 310° C. and an absolute pressure range of about 10 microns to about 25 microns.

In some embodiments, the estolide compounds and compositions may exhibit a kinematic viscosity of at least 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or even 350 cSt when measured at 100° C. In certain embodiments, the estolides exhibit a kinematic viscosity of about about 20 cSt to about 50 cSt at 100° C. In certain embodiments, the estolides exhibit a kinematic viscosity of about about 50 cSt to about 80 cSt at 100° C. In certain embodiments, the estolides exhibit a kinematic viscosity of about about 80 cSt to about 100 cSt at 100° C. In certain embodiments, the estolides exhibit a kinematic viscosity of about about 85 cSt to about 110 cSt at 100° C. In certain embodiments, the estolides exhibit a kinematic viscosity of about about 90 cSt to about 100 cSt at 100° C. In certain embodiments, the estolides exhibit a kinematic viscosity of about about 100 cSt to about 150 cSt at 100° C. In certain embodiments, the estolides exhibit a kinematic viscosity of about about 150 cSt to about 175 cSt at 100° C. In certain embodiments, the estolides exhibit a kinematic viscosity of about about 175 cSt to about 200 cSt at 100° C. In certain embodiments, the estolides exhibit a kinematic viscosity of about about 200 cSt to about 225 cSt at 100° C. In certain embodiments, the estolides exhibit a kinematic viscosity of about about 225 cSt to about 250 cSt at 100° C. In certain embodiments, the estolides exhibit a kinematic viscosity of about about 250 cSt to about 275 cSt at 100° C. In certain embodiments, the estolides exhibit a kinematic viscosity of about about 275 cSt to about 300 cSt at 100° C. In certain embodiments, the estolides exhibit a kinematic viscosity of about about 300 cSt to about 325 cSt at 100° C. In certain embodiments, the estolides exhibit a kinematic viscosity of about about 325 cSt to about 350 cSt at 100° C.

In some embodiments, the estolide compounds and compositions may exhibit a kinematic viscosity of at least 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, or even 3500 cSt when measured at 40° C. In certain embodiments, the estolides exhibit a kinematic viscosity of about about 200 cSt to about 500 cSt at 40° C. In certain embodiments, the estolides exhibit a kinematic viscosity of about about 500 cSt to about 800 cSt at 40° C. In certain embodiments, the estolides exhibit a kinematic viscosity of about about 800 cSt to about 1000 cSt at 40° C. In certain embodiments, the estolides exhibit a kinematic viscosity of about about 850 cSt to about 1100 cSt at 40° C. In certain embodiments, the estolides exhibit a kinematic viscosity of about about 900 cSt to about 1000 cSt at 40° C. In certain embodiments, the estolides exhibit a kinematic viscosity of about about 1000 cSt to about 1500 cSt at 40° C. In certain embodiments, the estolides exhibit a kinematic viscosity of about about 1500 cSt to about 2000 cSt at 40° C. In certain embodiments, the estolides exhibit a kinematic viscosity of about about 2000 cSt to about 2500 cSt at 40° C. In certain embodiments, the estolides exhibit a kinematic viscosity of about about 2500 cSt to about 3000 cSt at 40° C. In certain embodiments, the estolides exhibit a kinematic viscosity of about about 3000 cSt to about 3500 cSt at 40° C.

In certain embodiments, the estolides exhibit a kinematic viscosity of about about 400 cSt to about 800 cSt at 40° C. In certain embodiments, the estolides exhibit a kinematic viscosity of about about 400 cSt to about 600 cSt at 40° C. In certain embodiments, the estolides exhibit a kinematic viscosity of about about 425 cSt to about 550 cSt at 40° C. In certain embodiments, the estolides exhibit a kinematic viscosity of about about 450 cSt to about 500 cSt at 40° C. In certain embodiments, the estolides exhibit a kinematic viscosity of about about 460 cSt to about 480 cSt at 40° C. In certain embodiments, the estolides exhibit a kinematic viscosity of about about 550 cSt to about 750 cSt at 40° C. In certain embodiments, the estolides exhibit a kinematic viscosity of about about 600 cSt to about 725 cSt at 40° C. In certain embodiments, the estolides exhibit a kinematic viscosity of about about 650 cSt to about 700 cSt at 40° C. In certain embodiments, the estolides exhibit a kinematic viscosity of about about 1000 cSt to about 2000 cSt at 40° C. In certain embodiments, the estolides exhibit a kinematic viscosity of about about 1200 cSt to about 1800 cSt at 40° C. In certain embodiments, the estolides exhibit a kinematic viscosity of about about 1400 cSt to about 1600 cSt at 40° C.

In certain embodiments, estolides may exhibit desirable low-temperature pour point properties. In some embodiments, the estolide compounds and compositions may exhibit a pour point lower than about 0° C., about −5° C., about −10° C., about −15° C., about −20° C., about −25° C., about −30° C., about −35° C., −40° C., about −45° C., about −50° C., about −55° C., or even about about −60° C. In some embodiments, the estolide compounds and compositions have a pour point of about 0° C. to about −10° C. In some embodiments, the pour point falls within a range of about −5° C. to about −10° C., about −10° C. to about −20° C., about −20° C. to about −30° C., −30° C. to about −40° C., −40° C. to about −50, or about −50° C. to about −60° C.

In certain embodiments, the estolide compounds and compositions exhibit high viscosity indeces. For example, in certain embodiments, the estolides exhibit a viscosity of at least 150, 160, 170, 180, 190, 200, 205, 210, 215, 220, 225, or even 230. In certain embodiments, the estolides exhibit a viscosity index of about 150 to about 300, about 190 to about 300, about 160 to about 250, about 160 to about 180, about 180 to about 200, about 200 to about 210, about 210 to about 225, or about 225 to about 250.

In addition, in certain embodiments, the estolides may exhibit decreased Iodine Values (IV) when compared to estolides prepared by other methods. IV is a measure of the degree of total unsaturation of an oil, and is determined by measuring the amount of iodine per gram of estolide (cg/g). In certain instances, oils having a higher degree of unsaturation may be more susceptible to creating corrosiveness and deposits, and may exhibit lower levels of oxidative stability. Compounds having a higher degree of unsaturation will have more points of unsaturation for iodine to react with, resulting in a higher IV. Thus, in certain embodiments, it may be desirable to reduce the IV of estolides in an effort to increase the oil's oxidative stability, while also decreasing harmful deposits and the corrosiveness of the oil.

In some embodiments, estolide compounds and compositions described herein have an IV of less than about 40 cg/g or less than about 35 cg/g. In some embodiments, estolides have an IV of less than about 30 cg/g, less than about 25 cg/g, less than about 20 cg/g, less than about 15 cg/g, less than about 10 cg/g, or less than about 5 cg/g. The IV of a composition may be reduced by decreasing the estolide's degree of unsaturation. This may be accomplished by, for example, by increasing the amount of saturated capping materials relative to unsaturated capping materials when synthesizing the estolides. Alternatively, in certain embodiments, IV may be reduced by hydrogenating estolides having unsaturated caps.

Also described herein are methods of preparing high- and ultra high-viscosity estolide base oils and lubricants. Depending on the manner in which the estolide base oil is prepared, in certain embodiments it may not be possible to prepare high- and ultra high-viscosity estolide base oils using known methods and starting materials. In certain embodiments, estolides base oils exhibiting the desired characteristics may be prepared by a process that includes providing a first estolide base oil, and hydrolyzing the first estolide base oil to provide a product comprising at least one hydroxy fatty acid. In certain embodiments, the at least one hydroxy fatty acid may be subsequently oligomerized to provide an ultra high-viscosity estolide base oil.

In certain embodiments, the first estolide base oil can be prepared in any manner. However, in certain embodiments, it may be desirable to prepare the first estolide base oil from readily-available starting materials, including methods that include the use of unsaturated fatty acids. In certain embodiments, the first estolide base oil is prepared by a process that includes contacting at least one first fatty acid reactant with at least one second fatty acid reactant. In certain embodiments, the at least one first fatty acid reactant comprises at least one site of unsaturation. In certain embodiments, the at least one first fatty acid reactant comprises a free fatty acid. In certain embodiments, the at least one first fatty acid reactant comprises oleic acid, 9-decenoic acid, and/or 10-undecenoic acid. In certain embodiments, the at least one first fatty acid reactant comprises a fatty acid ester. In certain embodiments, the at least one first fatty acid reactant comprises methyl oleate. In certain embodiments, the first estolide base oil is prepared by a process that includes forming a covalent bond between an oxygen of a carboxylic group of at least one second fatty acid reactant and a carbon of at least one site of unsaturation of at least one first fatty acid reactnt.

In certain embodiments, it may be desirable to implement the use of a first estolide base oil that is readily hydrolyzed to provide the at least one hydroxy fatty acid. Without being bound to any particular theory, it is believed that estolide compounds comprising short-chain fatty acid caps may be more readily hydrolyzed than estolides comprising longer-chain fatty acid caps. Exemplary short-chain fatty acids may include C₁ to C₁₂ fatty acids, or even C₁ to C₈ fatty acids. Further, smaller estolide oligomers in their dimer or trimer form (e.g., EN=1 or 2) may be hydrolyzed more efficiently than larger oligomers under certain conditions. Thus, in certain embodiments, the first estolide base oil is prepared by contacting the at least one first fatty acid reactant (e.g., oleic acid) with a saturated fatty acid (e.g., acetic acid) in an effort to minimize oligomerization. In certain embodiments, the first estolide base oil the at least one first fatty acid reactant with a C₁ fatty acid, i.e., formic acid.

As noted above, in certain embodiments the first estolide base oil may be prepared by contacting oleic acid with a second fatty acid reactant. Because oleic acid comprises a double bond at the 9-postion of the fatty acid chain, hydrolysis of the first estolide base oil provides a fatty acid product that primarily comprises 9-hydroxystearic acid and 10-hydroxystearic acid. Depending on the catalyst and synthetic conditions implemented to effect estolide formation, isomerization of the oleic acid double bond may ultimately result in a hydrolyzed product comprising a minor amount of other hydroxystearic acids. In certain embodiments, hydrolysis may be effected by any suitable hydrolysis catalyst known to those of skill in the art, such as exposure to acidic (e.g., aqueous HCI) conditions, or basic (e.g., metal hydroxide such as KOH) conditions followed by acidification. In certain embodiments, hydrolysis of the first estolide base oil will yield the at least one hydroxy fatty acid and free fatty acids. In certain embodiments, the free fatty acids generated by hydrolysis represent the liberation of the second fatty acid reactant (e.g., acetic acid), which may be recovered and reused.

In certain embodiments, the at least one hydroxy fatty acid is oligomerized to provide a second estolide base oil. In certain embodiments, the second estolide base oil exhibits high or ultra high viscometric characteristics that make them suitable for certain applications. Without being bound to any particular theory, in certain embodiments it may be challenging to prepare estolides exhibiting desired high-viscosity characteristics using unsaturated fatty acid starting materials under certain conditions. Accordingly, in certain embodiments, high- and ultra-high viscosity estolide base oils may be prepared from reactive hydroxy fatty acid starting materials, such as 9-hydroxystearic acid and 10-hydroxystearic acid, via suitable condensation reaction conditions. Thus, in certain embodiments, the second estolide base oil is prepared by a process that includes forming a covalent bond between the carbon of a carboxylic acid group of a first hydroxy fatty acid and an oxygen of at least one hydroxy group of a second hydroxy fatty acid.

In certain embodiments, it may be be possible to maximize oligomerization conditions and, thus, the formation of high- and ultra high-viscosity estolides by purifying the hydroxy fatty acid feedstock. Without being bound to any particular theory, in certain embodiments it is believed that the oligomerization of hydroxy fatty acids may be limited due to the presence of certain “contaminants” in the feedstock stream. For example, in certain embodiments, some commercially-available hydroxy fatty acids may contain saturated fatty acids. Even in small concentrations, in certain embodiments, saturated fatty acids may prematurely cap a growing fatty acid oligomer, stunting the growth of the estolide molecule. Hence, in certain embodiments, providing a substantially pure hydroxy fatty acid feedstock, or a feedstock consisting essentially of hydroxy fatty acids, may be desirable for maximizing the size of the oligomer and, thus, the viscosity of the estolide base oil. In certain embodiments, the feedstock comprises greater than 85 or 90% hydroxy fatty acids by weight. In certain embodiments, the feedstock comprises greater than 90 or 95% hydroxy fatty acids by weight, such as about 98% or about 99%. In certain embodiments, the feedstock comprises about 90% to about 100% hydroxy fatty acids by weight, such as about 95% to about 99.9%.

In certain emobodiments, the first and second estolide base oils are formed from fatty acids in presence of any suitable catalyst known to those of skill in the art. In certain embodiments, the catalyst comprises one or more of a Bronsted Acid, a Lewis Acid, or dielectric heating. In certain embodiments, the catalyst comprises at least one of hydrochloric acid, nitric acid, methanesulfonic acid, sulfuric acid, phosphoric acid, perchloric acid, triflic acid, or p-TsOH. In certain embodiments, the catalyst comprises at least one of an acid-activated clay, a zeolite, or an acidic mesoporous material. In certain embodiments, the catalyst comprises at least one of a triflate salt or an iron compound. Exemplary catalysts include, but are not limited to, AgOTf, Cu(OTf)₂, Fe(OTf)₂, Fe(OTf)₃, NaOTf, LiOTf, Yb(OTf)₃, Y(OTf)₃, Zn(OTf)₂, Ni(OTf)₂, Bi(OTf)₃, La(OTf)₃, Sc(OTf)₃, Fe(acac)₃, FeC1₃, Fe₂(SO₄)₃, Fe₂O₃, and FeSO₄. Other exemplary catalysts may include tin compounds, hafnium compounds, titanium compounds, and zirconium compounds such as Sn(O₂CCO₂), SnO, SnCl₂, TiCl₄, Ti(OCH₂CH₂CH₂CH₃)₄, ZrCl₄, ZrOCl₂, ZrO(NO₃)₂, ZrO(SO₄), ZrO(CH₃COO)₂, ZrOCl₂.8H₂O, ZrOCl₂.2THF, HfCl₂, HfOCl₂, HfOCl₂.2THF and HfOCl₂.8H₂O.

The present disclosure further relates to methods of making estolides according to Formula I and II. By way of example, the reaction of an unsaturated fatty acid with an organic acid and the esterification of the resulting free acid estolide are illustrated and discussed in the following Schemes 1 and 2. The particular structural formulas used to illustrate the reactions correspond to those for synthesis of compounds according to Formula I; however, the methods apply equally to the synthesis of compounds according to Formula II, with use of compounds having structure corresponding to R₃ and R₄ with a reactive site of unsaturation.

As illustrated below, compound 100 represents an unsaturated fatty acid that may serve as the basis for preparing the estolide compounds described herein, such as a first estolide base il.

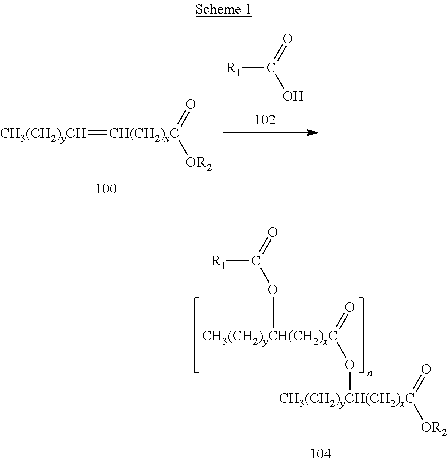

In Scheme 1, wherein x is, independently for each occurrence, an integer selected from 0 to 20, y is, independently for each occurrence, an integer selected from 0 to 20, n is an integer greater than or equal to 0, R₁ is selected from hydrogen and an optionally substituted alkyl that is saturated or unsaturated, and branched or unbranched, and R₂ is selected from hydrogen and an optionally substituted alkyl that is saturated or unsaturated, and branched or unbranched, unsaturated fatty acid reactant 100 may be combined with compound 102 and a catalyst to form estolide 104. In certain embodiments, compound 102 is not included, and unsaturated fatty acid reactant 100 comprises a free fatty acid (R₂=hydrogen) that may be exposed alone to catalytic conditions to form estolide 104, wherein R₁ would represent an unsaturated alkyl residue. In certain embodiments, fatty acid reactant 100 may comprise an unsaturated fatty acid ester (R₂=alkyl), which may minimize oligomerization and the size of estolide 104 (e.g., n=0 or 1), particularly when compound 102 is formic acid or a saturated fatty acid (R₁=hydrogen or alkyl). Thus, in certain embodiments, when compound 102 is included in the reaction, R₁ may represent hydrogen (formic acid) or one or more optionally substituted alkyl residues that are saturated or unsaturated and branched or unbranched (e.g., acetic acid or propionic acid). Any suitable catalyst may be implemented to catalyze the formation of estolide 104, including but not limited to Lewis acids, Bronsted acids, and/or dielectric heating (e.g., microwave radiation).

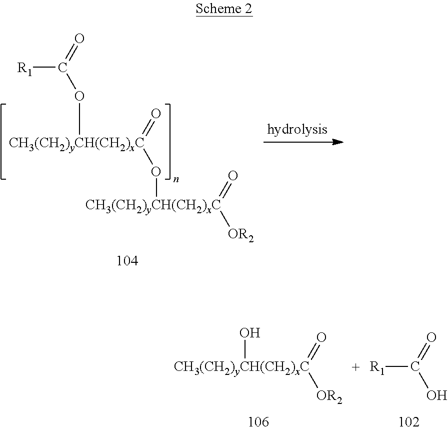

Similarly, in Scheme 2, wherein x is, independently for each occurrence, an integer selected from 0 to 20, y is, independently for each occurrence, an integer selected from 0 to 20, n is an integer greater than or equal to 0, and R₁ and R₂ are each independently selected from hydrogen and an optionally substituted alkyl that is saturated or unsaturated, and branched or unbranched, estolide 104 may be hydrolyzed using any suitable procedure known to those of skilled in the art, such as acid-catalyzed or base-catalyzed hydrolysis, to yield hydroxylated product 106 and regenerate compound 102. Depending on reaction conditions, when estolide 104 is esterified (R₂=alkyl), hydrolysis of estolide 104 may result in the hydrolytic cleavage of all ester bonds present in the molecule, such that resulting hydroxylated product 106 comprises a hydroxylated fatty acid (R₂=hydrogen).

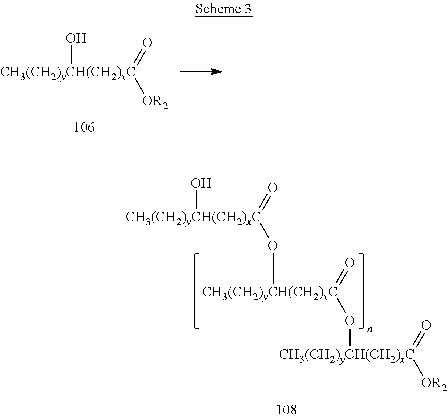

In Scheme 3 wherein x is, independently for each occurrence, an integer selected from 0 to 20, y is, independently for each occurrence, an integer selected from 0 to 20, n is an integer greater than or equal to 0, and R₂ is hydrogen, hydroxylated product 106 may be oligomerized using any suitable catalyst known to those of skill in the art to yield estolide 108 and water. Exemplary oligomerization catalysts include, but are not limited to, Bronsted acids such as hydrochloric acid, nitric acid, methanesulfonic acid, sulfuric acid, phosphoric acid, perchloric acid, triflic acid, or p-TsOH. The reaction may also be catalyzed by one or more Lewis acids selected from tin compounds, zirconium compounds, hafnium compounds, and titanium compounds.

As discussed above, in certain embodiments, the estolides described herein may have improved properties which render them useful as base stocks for biodegradable lubricant applications. Such applications may include, without limitation, crankcase oils, gearbox oils, hydraulic fluids, drilling fluids, two-cycle engine oils, greases, and the like. Other suitable uses may include marine applications, where biodegradability and toxicity are of concern. In certain embodiments, the nontoxic nature of certain estolides described herein may also make them suitable for use as lubricants in the cosmetic and food industries.

In certain embodiments, the estolide compounds may meet or exceed one or more of the specifications for certain end-use applications, without the need for conventional additives. For example, in certain instances, high-viscosity lubricants, such as those exhibiting a kinematic viscosity of greater than about 120 cSt at 40° C., or even greater than about 200 cSt at 40° C., may be desired for particular applications such as gearbox or wind turbine lubricants. Prior-known lubricants with such properties typically also demonstrate an increase in pour point as viscosity increases, such that prior lubricants may not be suitable for such applications in colder environments.

However, in certain embodiments, the counterintuitive properties of certain compounds described herein (e.g., increased EN provides estolides with higher viscosities while retaining, or even decreasing, the oil's pour point) may make higher-viscosity estolides particularly suitable for such specialized applications.

Similarly, the use of prior-known lubricants in colder environments may generally result in an unwanted increase in a lubricant's viscosity. Thus, depending on the application, it may be desirable to use lower-viscosity oils at lower temperatures. In certain circumstances, low-viscosity oils may include those exhibiting a viscosity of lower than about 50 cSt at 40° C., or even about 40 cSt at 40° C. Accordingly, in certain embodiments, the low-viscosity estolides described herein may provide end users with a suitable alternative to high-viscosity lubricants for operation at lower temperatures.

In some embodiments, it may be desirable to prepare lubricant compositions comprising an estolide base stock. For example, in certain embodiments, the estolides described herein may be blended with one or more additives selected from polyalphaolefins, synthetic esters, polyalkylene glycols, mineral oils (Groups I and II), pour point depressants, viscosity modifiers, anti-corrosives, antiwear agents, detergents, dispersants, colorants, antifoaming agents, and demulsifiers. In addition, or in the alternative, in certain embodiments, the estolides described herein may be co-blended with one or more synthetic or petroleum-based oils to achieve desired viscosity and/or pour point profiles. In certain embodiments, certain estolides described herein also mix well with gasoline, so that they may be useful as fuel components or additives.

In all of the foregoing examples, the compounds described may be useful alone, as mixtures, or in combination with other compounds, compositions, and/or materials.

Methods for obtaining the novel compounds described herein will be apparent to those of ordinary skill in the art, suitable procedures being described, for example, in the examples below, and in the references cited herein.

### EXAMPLES

### Analytics

Nuclear Magnetic Resonance: NMR spectra were collected using a Bruker Avance 500 spectrometer with an absolute frequency of 500.113 MHz at 300 K using CDCl₃ as the solvent. Chemical shifts were reported as parts per million from tetramethylsilane. The formation of a secondary ester link between fatty acids, indicating the formation of estolide, was verified with ¹H NMR by a peak at about 4.84 ppm.

Estolide Number (EN): The EN was measured by GC analysis. It should be understood that the EN of a composition specifically refers to EN characteristics of any estolide compounds present in the composition. Accordingly, an estolide composition having a particular EN may also comprise other components, such as natural or synthetic additives, other non-estolide base oils, fatty acid esters, e.g., triglycerides, and/or fatty acids, but the EN as used herein, unless otherwise indicated, refers to the value for the estolide fraction of the estolide composition.

Iodine Value (IV): The iodine value is a measure of the degree of total unsaturation of an oil. IV is expressed in terms of centigrams of iodine absorbed per gram of oil sample. Therefore, the higher the iodine value of an oil the higher the level of unsaturation is of that oil. The IV may be measured and/or estimated by GC analysis. Where a composition includes unsaturated compounds other than estolides as set forth in Formula I and II, the estolides can be separated from other unsaturated compounds present in the composition prior to measuring the iodine value of the constituent estolides. For example, if a composition includes unsaturated fatty acids or triglycerides comprising unsaturated fatty acids, these can be separated from the estolides present in the composition prior to measuring the iodine value for the one or more estolides.

Acid Value: The acid value is a measure of the total acid present in an oil. Acid value may be determined by any suitable titration method known to those of ordinary skill in the art. For example, acid values may be determined by the amount of KOH that is required to neutralize a given sample of oil, and thus may be expressed in terms of mg KOH/g of oil.

Gas Chromatography (GC): GC analysis was performed to evaluate the estolide number (EN) and iodine value (IV) of the estolides. This analysis was performed using an Agilent 6890N series gas chromatograph equipped with a flame-ionization detector and an autosampler/injector along with an SP-2380 30 m×0.25 mm i.d. column.

The parameters of the analysis were as follows: column flow at 1.0 mL/min with a helium head pressure of 14.99 psi; split ratio of 50:1; programmed ramp of 120-135° C. at 20° C./min, 135-265° C. at 7° C./min, hold for 5 min at 265° C.; injector and detector temperatures set at 250° C.

Measuring EN and IV by GC: To perform these analyses, the fatty acid components of an estolide sample were reacted with MeOH to form fatty acid methyl esters by a method that left behind a hydroxy group at sites where estolide links were once present. Standards of fatty acid methyl esters were first analyzed to establish elution times.

Sample Preparation: To prepare the samples, 10 mg of estolide was combined with 0.5 mL of 0.5M KOH/MeOH in a vial and heated at 100° C. for 1 hour. This was followed by the addition of 1.5 mL of 1.0 M H₂SO₄/MeOH and heated at 100° C. for 15 minutes and then allowed to cool to room temperature. One (1) mL of H₂O and 1 mL of hexane were then added to the vial and the resulting liquid phases were mixed thoroughly. The layers were then allowed to phase separate for 1 minute. The bottom H₂O layer was removed and discarded. A small amount of drying agent (Na₂SO₄ anhydrous) was then added to the organic layer after which the organic layer was then transferred to a 2 mL crimp cap vial and analyzed.

EN Calculation: The EN is measured as the percent hydroxy fatty acids divided by the percent non-hydroxy fatty acids. As an example, a dimer estolide would result in half of the fatty acids containing a hydroxy functional group, with the other half lacking a hydroxyl functional group. Therefore, the EN would be 50% hydroxy fatty acids divided by 50% non-hydroxy fatty acids, resulting in an EN value of 1 that corresponds to the single estolide link between the capping fatty acid and base fatty acid of the dimer.

IV Calculation: The iodine value is estimated by the following equation based on ASTM Method D97 (ASTM International, Conshohocken, Pa.):

$$IV = Σ100 \times \frac{{A}_{f} \times {MW}_{I} \times db}{{MW}_{f}}$$

A_{f}=fraction of fatty compound in the sample
MW_{I}=253.81, atomic weight of two iodine atoms added to a double bond
db=number of double bonds on the fatty compound
MW_{f}=molecular weight of the fatty compound

The properties of exemplary estolide compounds and compositions described herein are identified in the following examples and tables.

Other Measurements: Except as otherwise described, pour point is measured by ASTM Method D97-96a, cloud point is measured by ASTM Method D2500, viscosity/kinematic viscosity is measured by ASTM Method D445-97, viscosity index is measured by ASTM Method D2270-93 (Reapproved 1998), specific gravity is measured by ASTM Method D4052, flash point is measured by ASTM Method D92, evaporative loss is measured by ASTM Method D5800, vapor pressure is measured by ASTM Method D5191, and acute aqueous toxicity is measured by Organization of Economic Cooperation and Development (OECD) 203.

### Example 1

A glass-lined reactor equipped with an agitator, thermocouple, and nitogen inlet, was purged with nitrogen to maintain a nitrogen atmosphere at all times. The reactor was charged with high oleic acid feed (4.29 kg, 1.0 eq, 15.17 moles), which was agitated at 200-300 rpm. Next, glacial acetic acid (9.15 kg, 10.0 eq, 152.37 moles) was added to the reactor. The reactor was cooled using a water bath, and triflic acid (0.47 kg, 0.20 eq, 3.14 moles) was slowly added to the reactor. The reactor was then heated at 60° C. for 24-72 hrs, with completion of the reaction being monitored by TLC. The reactor was placed under vacuum (40-200 mbar) at 40-60° C. to remove any unreacted acetic acid.

The temperature of the reaction vessel was maintained at 60° C., and tap water (1.2 kg) was slowly added. An aqueous solution of KOH (86% purity, 3.44 kg, 52.67 moles, 3.47 equivalents) in tap water (6 kg) was slowly added to the reaction mixture. The reaction vessel was then heated to 85-90° C. for about 2 hrs, with completion of the hydrolysis being monitored by TLC. The crude reaction mixture was then transferred to a reactor containing sulfuric acid (2.13 kg) and tap water (2.13 kg) such that the temperature remained <90° C. The reaction mixture was then maintained at a temperature of 75-85° C., and washed with hot tap water (75-85° C., 4×12 kg) until the final aqueous wash had a pH of 4.5-6. Several additional water washes were used as needed to ensure that the inorganic salts were extracted. Water present in the reactor was then removed under vacuum (40-80 mbar) at 85-90° C. to yield a crude reaction mixture of 9-hydroxystearic acid and 10-hydroxystearic acid.

The temperature of the crude reaction mixture was maintained at 85-90° C., and heptanes (3.86 kg, 5.65 L) were added. The mixture was allowed to cool to ambient temperature, and was stirred overnight. The mixture may be allowed to stir overnight at room temperature. The mother liquor was then siphoned from the reaction mixture and discarded. This purification was repeated twice more using heptanes (2.74 kg, 4.00 L). At room temperature, the remaining slurry was filtered off over a fritted funnel. The wetcake was washed with heptanes (7×0.48 kg). The wetcake solids were allowed to dry at ambient temperature and atmospheric pressure to provide a mixture of 9/10-hydroxystearic acids.

### Example 2

A reaction vessel purged with was charged 9/10-hydroxystearic acids (1.56 kg, 5.00 moles, 1.0 eq) produced according to the method of Example 1. The reactor was heated to 85-90° C. and the reaction mixture was stirred until complete dissolution was achieved. The temperature of the reactor was maintained at 85-90° C., and methanesulfonic acid (12.50 g, 0.13 moles, 0.025 eq) was added. The reactor was then placed under vacuum (40-80 mbar) and stirred at 100° C. for about 24 hrs. During this step, water distillate was collected in a cooled receiver flask. The reaction mixture was allowed to react until water distillate ceased to collect in the distillate receiver.

The temperature of the reaction mixture maintained at 60-80° C., and 2-ethylhexanol (0.27 kg, 2.05 moles, 0.39 eq) was added. The reactor was then placed under vacuum (40-80 mbar) at 60-70° C. for 3 to 6 hrs. Samples of the reaction mixture were taken to determine Total Acid Number (TAN), and the reaction was only allowed to proceed to the next step when TAN was ≦4 mg KOH/g (uncorrected for MSA) or ≦0.5 mg KOH/g (corrected for MSA).

### Example 3

The crude reaction mixture from Example 2 at room temperature was charged with Amberlite IRA-402(OH) resin (water wet, 0.41 kg, 23 wt % loading) and heptanes (0.17 kg) the mixture to stir at ambient temperature for about 24 hrs, and was then polish filtered over a coarse fritted funnel containing Celite (0.12 kg). The filtered crude estolide base oil was then charged to a reactor and agitated at 150-250 rpm. The reactor was placed under vacuum pressure (40-80 mbar) and agitated at 40-145° C. to remove excess 2-ethylhexanol/heptanes and water. The resulting residue was then placed in a reactor charged with nitrogen, and agitated at 150-250 rpm. The reactor was charged with Amberlite IRA-402(OH) resin (methanol washed and atmospherically dried, 21 wt % loading) and heptanes (25 wt % loading), and stirred at ambient temperature for about 24 hrs. The resulting mixture was then polish filtered over a coarse fritted funnel containing Celite (5-10 wt % loading). Filtration of about 1 kg of high viscosity estolide over Celite required about 1.5 hrs. Remaining heptanes were removed by distillation at 40° C. under high vacuum (600-2000 micron).

### Example 4

A glass-lined reactor equipped with an agitator, thermocouple, and nitogen inlet, was purged with nitrogen to maintain a nitrogen atmosphere at all times. The reactor was charged with high oleic acid feed (4.29 kg, 1.0 eq, 15.17 moles) and agitated at 200-300 rpm. The reactor was then charged with glacial acetic acid (9.15 kg, 10.0 eq, 152.37 moles). Triflic acid (0.47 kg, 0.20 eq, 3.14 moles) was then added, using a water bath (cold city water) to maintain the ambient temperature of the reaction mixture. The reactor was then heated to 60° C. under nitrogen and stirred 24-72 hrs. Completion of the reaction was confirmed by TLC, and the reactor was placed under vacuum (40-200 mbar) at 40-60° C. to remove unreacted acetic acid.

The crude reaction mixture (dark brown) was maintained at 60° C., and then tap water (1.2 kg) was slowly added. The reactor was then slowly charged with a solution of potassium hydroxide (86% purity, 3.44 kg, 52.67 moles, 3.47 equivalents) in tap water (6.0 kg). The reaction mixture was then heated under nitrogen with agitation at 85-90° C. for about 2 hours, with completion of the reaction being monitored by HPLC. The alkaline reaction mixture was then transferred to a reactor containing sulfuric acid (2.13 kg) and tap water (2.13 kg) such that the temperature remained ≦90 oC. The reaction mixture temperature was maintained at 75-85° C., and washed with hot tap water (75-85 oC, 4×12 kg) until the final aqueous wash had a pH of 4.5-6. Additional water washes were used as needed to ensure that the inorganic salts are extracted. The reactor was then placed under vacuum (40-80 mbar) at 85-90° C. to remove the water to yield a crude reaction mixture of 9-hydroxystearic acid and 10-hydroxystearic acid.

The temperature of the crude reaction mixture was maintained at 85-90° C., and heptanes (3.86 kg, 5.65 L) were added. The reacton mixture was allowed to cool to ambient temperature, and was stirred overnight at room temperature. The mother liquor was then siphoned from the reaction mixture and discarded. This purification was repeated twice more using heptanes (2.74 kg, 4.00 L). At room temperature, the resulting slurry was filtered over fritted funnel. The wetcake was washed with heptanes (7×0.48 kg), and the wetcake solids were dried at ambient temperature and atmospheric pressure to provide a mixture of 9/10-hydroxystearic acids.

### Example 5

A glass-lined reactor equipped with an agitator, thermocouple, and nitogen inlet, was purged with nitrogen to maintain a nitrogen atmosphere at all times. The reactor was charged with 9/10-hydroxystearic acids having a purity of >99% (1.56 kg, 5.00 moles, 1.0 eq) prepared according to the method set forth in Example 5. The reactor was heated to 85-90° C. and stirred until complete dissolution is achieved. The reaction mixture was maintained at 85-90° C., and the vessel was charged methanesulfonic acid (12.50 g, 0.13 moles, 0.025 eq). Under stirring the reaction vessel was placed under vacuum (40-80 mbar) at 100° C. for about 24 hrs. During this step, water distillate was collected in a cooled receiver flask. The reaction was continued until water distillate collection ceased.

The crude reaction mixture was then warmed to 60-80° C., and the reaction vessel was charged 2-ethylhexanol (0.27 kg, 2.05 moles, 0.39 eq) and placed under vacuum (40-80 mbar) at 60-70° C. for 3 to 6 hours. Total Acid Number (TAN) of the reaction was monitored until a TAN of <4 mg KOH/g (uncorrected for MSA) or <0.5 mg KOH/g (corrected for MSA) was achieved. Typical TAN results: 3.79 mg KOH g-1 (uncorrected for MSA) 0.299 mg KOH/g (corrected for MSA). At room temperature, the reaction vessel was charged with Amberlite IRA-402(OH) resin (methanol washed and atmospherically dried, 0.14 kg, 7-10 wt % loading). The mixture was allowed to stir at ambient temperature for about 24 hours, and was then polish filtered over a coarse fritted funnel containing Celite to provide the crude estolide base oil.

### Example 6

The crude estolide base oil of Example 5 was distilled using a wiped-film still unit 75-90° C. under high vacuum pressure (100-300 micron). The resulting oil was then added to a nitrogen-purged reactor and agitated at 150-250 rpm. The reactor was then charged with Amberlite IRA-402(OH) resin (methanol washed and atmospherically dried, 21 wt % loading) and heptanes (25 wt % loading). The reaction mixture was allowed to stir at ambient temperature for about 24 hrs, and was then polish filtered over a coarse fritted funnel containing Celite (5-10 wt % loading). Filtration of about 1 kg of high viscosity estolide over Celite required 1.5 hours to achieve complete filtration. The heptanes by distillation at 40° C. under high vacuum (600-2000 micron) to provide the purified high-viscosity estolide base oil. The properties of the resulting product are set forth below in Table 1:

| | | Unit of | |
|---|---|---|---|
| Property | Method | Measure | Result |
| KV 40° C. | D445 | cSt | 2389.2 |
| KV 100° C. | D445 | cSt | 218.3 |
| VI | D2270 | — | 224 |
| Pour Point | D97 | ° C. | −21 |
| Cloud Point | D2500 | ° C. | −29 |
| TAN | D664 | mg KOH/g | 0.07 |
| Hydroxyl Value | D1957 | mg KOH/g | 14.7 |
| Flash Point | D92 | ° C. | 291 |
| Fire Point | D92 | ° C. | 303 |
| RPVOT¹ | D2272 | min | 1852 |
| Wear Scar (4BW)² | D4172 | mm | 0.41 |
| Hydrolytic Stability (144 h) | D2619 | mg KOH/g | 7.61 |
| Biodegradability | OECD | % | 58.8 |
| | 301B | | |

¹Formulated with BT's standard 1 wt % antioxidant package (1:1 ratio of phenolic/aminic AO).
²1200 rpm, 40 kgf, 75 C., 1 h

### Example 7

Estolides wer prepared according to the method set forth in Example 5, except the 9/10-hydroxystearic acids having a purity of >99% were replaced with a 12-hydroxystearic acid feedstock having a purity of about 85%. The resulting crude estolide base oil was purified according to the method set forth in Example 6 to provide a purified high-viscosity estolide base oil having the following properties:

| | | Unit of | |
|---|---|---|---|
| Property | Method | Measure | Result |
| KV 40° C. | D445 | cSt | 522.9 |
| KV 100° C. | D445 | cSt | 64.1 |
| VI | D2270 | — | 197 |
| Pour Point | D97 | ° C. | −18 |
| Cloud Point | D2500 | ° C. | −6 |
| TAN | D664 | mg KOH/g | 0.12 |
| Hydroxyl Value | D1957 | mg KOH/g | 7.5 |
| Flash Point | D92 | ° C. | 293 |
| Fire Point | D92 | ° C. | 307 |
| RPVOT¹ | D2272 | min | 953 |
| Wear Scar (4BW)² | D4172 | mm | 0.44 |
| Hydrolytic Stability | D2619 | mg KOH/g | 4.83 |
| (144 h) | | | |

¹Formulated with BT's standard 1 wt % antioxidant package (1:1 ratio of phenolic/aminic AO).
²1200 rpm, 40 kgf, 75 C., 1 h

1-224. (canceled)

225. A composition comprising one or more compounds of Formula I, said composition having an EN selected from an integer or fraction of an integer that is equal to or greater than 5, wherein the EN is the average number of estolide linkages in compounds according to Formula I, and a kinematic viscosity selected from 425 to 550 cSt when measured at 40° C., wherein the one or more compounds are selected from: 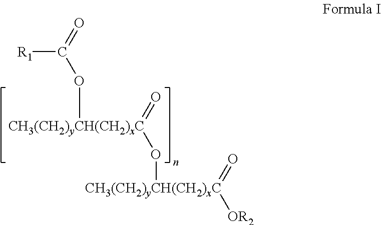
wherein
x is, independently for each occurrence, an integer selected from 0 to 20;
y is, independently for each occurrence, an integer selected from 0 to 20;
n is equal to or greater than 0;
R₁ is an optionally substituted alkyl that is saturated or unsaturated, and branched or unbranched; and
R₂ is an optionally substituted alkyl that is saturated or unsaturated, and branched or unbranched,
wherein each fatty acid chain residue of said at least one compound is independently optionally substituted.

226. The composition according to claim 225, wherein
x is, independently for each occurrence, an integer selected from 0 to 14;
y is, independently for each occurrence, an integer selected from 0 to 14;
n is an integer selected from 0 to 20;
R₁ is an optionally substituted C₁ to C₂₂ alkyl that is saturated or unsaturated, and branched or unbranched; and
R₂ is an optionally substituted C₁ to C₂₂ alkyl that is saturated or unsaturated, and branched or unbranched, wherein each fatty acid chain residue is unsubstituted.

227. The composition according to claim 226, wherein n is an integer selected from 5 to 15.

228. The composition according to claim 226, wherein EN is selected from 5 to 20.

229. The composition according to claim 226, wherein
x+y is, independently for each chain, an integer selected from 13 to 15; and
n is an integer selected from 0 to 12.

230. The composition according to claim 228, wherein R₂ is a branched or unbranched C₁ to C₂₀ alkyl that is saturated.

231. The composition according to claim 228, wherein R₂ is selected from branched C₆ to C₁₂ alkyl.

232. The composition according to claim 231, wherein R₂ is 2-ethylhexyl.

233. The composition according to claim 228, wherein R₁ is selected from unsubstituted C₁ to C₂₀ alkyl that is unbranched and saturated or unsaturated.

234. The composition according to claim 233, wherein R₁ is saturated.

235. The composition according to claim 228, wherein x is, independently for each occurrence, selected from 7 to 10.

236. The composition according to claim 228, wherein x is, independently for each occurrence, selected from 7 and 8.

237. The composition according to claim 235, wherein y is, independently for each occurrence, selected from 7 and 8.

238. The composition according to claim 235, wherein y is, independently for each occurrence, selected from 5 to 8.

239. The composition according to claim 236, wherein y is 0 for each occurrence.

240. The composition according to claim 237, wherein said composition has a kinematic viscosity of 450 cSt to 500 cSt at 40° C.

241. The composition according to claim 226, wherein said composition exhibits an iodine value (IV) of less than 10 cg/g.

242. The composition according to claim 226, wherein said composition exhibits an iodine value (IV) of less than 5 cg/g.

243. A method comprising contacting a gearbox with the composition according to claim 228.

244. A method comprising contacting a wind turbine with the composition according to claim 228.
